(19) United States
(12) Patent Application Publication
(10) Pub. No.: US 2025/0232299 A1
(43) Pub. Date: Jul. 17, 2025

(54) METHOD AND APPARATUS FOR VERIFYING TRANSACTION IN METAVERSE ENVIRONMENT

(71) Applicant: Samsung Electronics Co., Ltd., Suwon-si (KR)

(72) Inventors: Sandeep Singh SPALL, Noida (IN); Choice CHOUDHARY, Noida (IN); Amitoj SINGH, Noida (IN)

(21) Appl. No.: 19/069,751

(22) Filed: Mar. 4, 2025

Related U.S. Application Data
(63) Continuation of application No. PCT/KR2022/019059, filed on Nov. 29, 2022.

(30) Foreign Application Priority Data
Sep. 8, 2022 (IN) 202241051269

(51) Int. Cl. G06Q 20/40; A61B 5/00; A61B 5/11; A61B 5/16; A61B 5/245; A61B 5/372; A61B 5/375; G06Q 50/00; G10L 25/18; G10L 25/30; G10L 25/51

(52) U.S. Cl. CPC G06Q 20/401; A61B 5/1118; A61B 5/165; A61B 5/245; A61B 5/372; A61B 5/375; A61B 5/7246; A61B 5/7264; A61B 5/746; G06Q 50/01; G10L 25/51; A61B 2503/12; G10L 25/18; G10L 25/30

(57) ABSTRACT

A method for verifying transactions in a metaverse environment is disclosed. The method comprises: detecting a first user involved in an activity using user biometrics, wherein the first user is in focused attention state or addiction state; identifying a second user interacting with the first user during a transaction; identifying an intention of the second user interacting with the first user; determining a presence of an abnormality in a physiological state of the first user; and recommending to the first user to focus on the transaction.

### CROSS-REFERENCE TO RELATED APPLICATIONS

This application is a continuation of International Application No. PCT/KR2022/019059 designating the United States, filed on Nov. 29, 2022, in the Korean Intellectual Property Receiving Office and claiming priority to Indian Patent Application No. 202241051269, filed on Sep. 8, 2022, in the Indian Patent Office, the disclosures of each of which are incorporated by reference herein in their entireties.

### BACKGROUND

### Field

The disclosure relates to a system to verify transactions in a metaverse environment for preventing and/or reducing physiological tricks and method thereof. For example, the disclosure relates to a system and method for verifying transactions between a first user and a second user, wherein the system determines the intent of the second user to identify the abnormalities in the initiated transaction and provide recommendations to the first user by prompting for physical verification of the transaction.

### Description of Related Art

The metaverse environment may refer to a shared, realistic, and immersive computer simulation of the real-world environment in which users participate as digital avatars. The metaverse allows the users to perform a variety of activities including working, meeting, gaming, and socializing with other users in three-dimensional spaces. The applications of the metaverse are increasing day-by-day, leading to the linking of the physical and virtual worlds with the financial world. Though the usage of metaverse environment has proved to be useful to carry out various activities, the routine usage may make the user suffer from physical and mental harassment which makes user health biomarkers abnormal e.g., online transaction scams, scams caused by hypnotization. By analyzing various user related parameters and enhancing the user experience for the user by providing more secured features while the user is in metaverse is not explored yet.

The existing metaverse platforms provide the users a platform for socializing, learning, collaborating, playing, and completing various financial transactions, which involve the use of digital currency such as Bitcoin. The payment transaction system in the metaverse environment includes the identification of people and businesses, determination of goods or services, and the consensus on transaction information. With the help of Non-Fungible Tokens (NFT) and blockchain system, the transactions are processed and using a verification system, the user is verified.

The increase in financial transactions in the metaverse environment has led to a breach of the security of the users, wherein social engineering is used for psychological manipulation of people into performing actions or divulging confidential information, which may involve network-based attacks to retrieve information from a user and by human-based attacks where users are manipulated into revealing sensitive information. By deceiving and manipulating human psychology, the attackers trick their victims into taking actions on behalf of the attacker and then obtain sensitive information. While the user experiences focused attention or addiction by turning off any background noises, the user is considered to enter a state of trance, which makes it easy for the attackers to deceive the users in the metaverse environment.

For instance, U.S. Pat. No. 8,099,668B2 titled “Predator and abuse identification and prevention in a virtual environment” discloses systems and techniques for protecting a child user from inappropriate interactions within an immersive virtual environment, where inappropriate interactions may be detected by examining characteristics of the interactions between a child and another user (e.g., communications, transactions, etc.), by monitoring physical signs of stress in the child or by receiving software commands given by the child to signal discomfort in a particular situation and all financial transactions of the child will be blocked. Subsequently, preventative actions may be determined based on a level of severity of the inappropriate interaction. The system allows no financial transactions and whenever inappropriate interactions may be detected by examining characteristics of the interactions between a child and another user. However, the disclosure in “U.S. Pat. No. 8,099,668B2” is limited to protecting a child user from inappropriate interactions using stress detection.

For instance, U.S. Pat. No. 9,819,711B2 titled “Online social interaction, education, and health care by analyzing affect and cognitive features” discloses a method of establishing a collaborative platform for Online social interaction, education, and health care by analyzing the effect and cognitive features of the individuals. The interactions between two users are analyzed using facial features, time and location, typing and extraction of audio features to determine the user's mental and emotional state. A collaborative interactive session is performed for a plurality of members, and the effect and/or cognitive features of some or all of the plurality of members are analyzed. A unified collaborating platform is created for social interaction, education, healthcare, gaming, etc. that allows users of any social media service to interact. The emotional and mental analysis of the interaction between users will then be used for targeted advertisements and for creating a smart e-learning system. However, the disclosure in “U.S. Pat. No. 9,819,711B2” is limited to the creation of a collaborative interactive platform and determination of the mental and emotional state of users for targeted ads and smart e-learning purposes

For instance, U.S. Pat. No. 10,726,465B2 titled “System, method and computer program product providing eye tracking based cognitive filtering and product recommendations” discloses techniques related to product recommendations, where biometric data (e.g., eye movement data) is used when a user selects displayed items for purchase and recommends other products. Such techniques provide a method to input biometric data (eye) generated for a user in an environment into a data processing system, wherein the user selects one or more displayed items for purchase, and then the method recommends other items not selected as being of potential interest. However, the disclosure in “U.S. Pat. No. 10,726,465B2” is limited to recommending not selected products by users, using eye biometric data, during purchase.

For instance, the publication titled “Detection of online harassment which users can face in social networks” relates to text analysis, preprocessing, and person identification, to determine online harassment using a pattern-based approach. The system detects online harassment by analyzing text to identify content that might cause psychological harm and then detect links between such content and references to a person. The modules are further organized in a three-step process consisting of text preprocessing, person identification, and classification. The system focuses on determining online harassment along with person identification by analysing text using a pattern-based approach.

Hence, there exists a need for a system to verify the transactions between a first user and a second user in a metaverse environment, in real-time.

### SUMMARY

Embodiments of the disclosure address the drawbacks of the prior art and provide a system to verify transactions associated with a user, while the user is in focused attention or addiction state, in a metaverse environment, wherein the verification of transaction facilitates the prevention/reduction of physiological tricks. The transactions between a first user and a second user are verified by detecting the presence of the first user involved in an activity using the user's biometrics, wherein the first user is in focused attention or addiction state. Further, the intention of the second user interacting with the first user is determined, wherein the intention of the second user interacting with the first user is identified through an intention finder module using artificial intelligence and deep learning techniques.

The intention of the second user is correlated with the current physiological state of the first user using the user's biometrics, wherein the correlation includes variations in the physiological state of the first user captured by a physical activity sensor module and a brain activity sensor module. Upon correlating the intention of the second user with the physiological state of the first user, the presence of any abnormality in the physiological state of the first user is determined by a processing module, and the authenticity of the transaction is determined by a transaction classifier. Further, a recommendation module recommends the first user to focus on the transaction.

Embodiments of the disclosure provide a system to verify transactions in a metaverse environment, wherein the system comprises an activator module for monitoring initiation of a transaction from the first user, and the physical activity of the first user is determined by the physical activity sensor module. The brain activity of the first user is monitored using a brain activity sensor module, wherein the physiological state of the first user is determined. Further, the presence of any physical activity in the first user is identified by a processing module, wherein the active attention state, physiological state, and presence of any disorder in the first user are identified.

According to an example embodiment of the present disclosure, a method for verifying transactions in a metaverse environment is disclosed. The method may comprise: detecting a first user involved in an activity using user biometrics, wherein the first user is in a focused attention state or addiction state; identifying a second user interacting with the first user during a transaction; identifying an intention of the second user interacting with the first user; determining a presence of an abnormality in a physiological state of the first user; and recommending to the first user to focus on the transaction.

According to an example embodiment of the present disclosure, an electronic device for verifying transactions in a metaverse environment is disclosed. The electronic device may comprise: a memory and at least one processor, comprising processing circuitry, coupled to the memory, wherein at least one processor, individually and/or collectively, may be configured to: detect a first user involved in an activity using user biometrics, wherein the first user is in a focused attention state or addiction state; identify a second user interacting with the first user during a transaction; identify an intention of the second user interacting with the first user; determine a presence of an abnormality in a physiological state of the first user; and recommend to the first user to focus on the transaction.

### BRIEF DESCRIPTION OF THE DRAWINGS

In the drawings, like reference numerals refer to like elements. The above and other aspects, features and advantages of certain embodiments of the present disclosure will be more apparent from the following detailed description, taken in conjunction with the accompanying drawings, in which:

FIG. 1 is a flowchart illustrating an example method to verify transactions in the metaverse environment according to various embodiments;

FIG. 2 is a block diagram illustrating an example configuration of a system to verify transactions in the metaverse environment according to various embodiments;

FIG. 3 is a block diagram illustrating an example configuration of the brain activity sensor module according to various embodiments;

FIG. 4 is a block diagram illustrating an example of the activity recognition by the activity detection unit according to various embodiments;

FIG. 5 is a block diagram illustrating an example configuration of the focused attention unit according to various embodiments;

FIG. 6 is a block diagram illustrating an example configuration of the emotion detection unit according to various embodiments;

FIG. 7 is a block diagram illustrating an example configuration of the classification unit of the emotion detection unit according to various embodiments;

FIG. 8 is a block diagram illustrating an example configuration of the disorder detection unit according to various embodiments;

FIG. 9 is a block diagram illustrating an example configuration of the intention finder module according to various embodiments;

FIG. 10 is a flowchart illustrating an example operation of a first use case according to various embodiments; and

FIG. 11 is a flowchart illustrating an example operation of a second use case according to various embodiments.

### DETAILED DESCRIPTION

Reference will now be made in detail to the description of the present subject matter, one or more examples of which are shown in figures. Each example is provided to explain the subject matter and is not to be considered a limitation. Various changes and modifications apparent to one skilled in the art to which the disclosure pertains are deemed to be within the spirit, scope and contemplation of the disclosure.

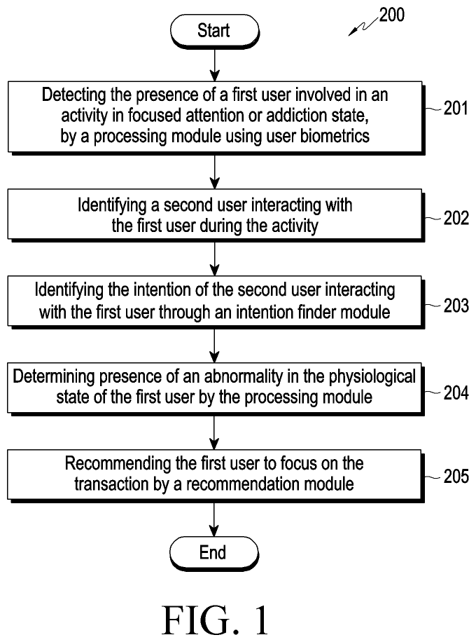

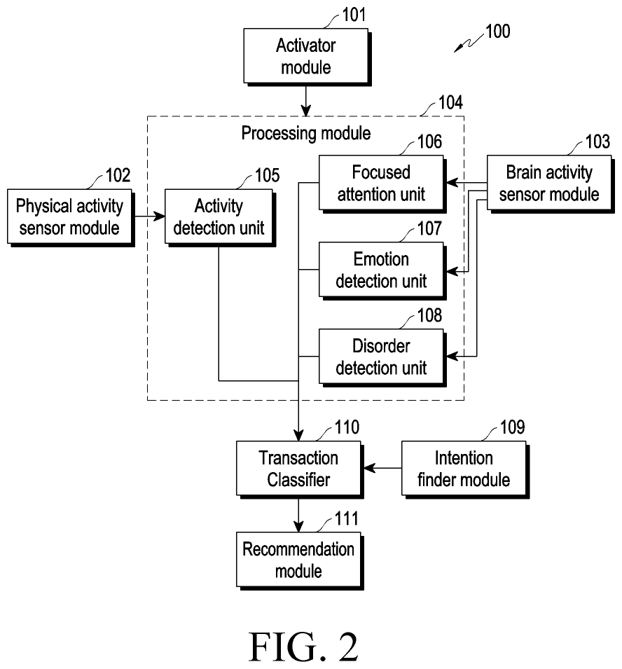

FIG. 1 is a flowchart illustrating an example method to verify transactions in the metaverse environment, according to various embodiments; The method (200) comprises detecting the presence of a first user involved in an activity using user biometrics through a processing module (e.g., 104 of FIG. 2), in step (201), wherein the first user is immersed in focused attention or addiction state.

In step (202), a second user interacting with the first user during the activity is identified, wherein the interaction may include a transaction or a physiological trick. Further, the intention of the second user interacting with the first user during the interaction may be identified using an intention finder module (e.g., 109 of FIG. 2), in step (203), the intention of the second user is identified using artificial intelligence, based on detection of the user sentiment from the voice of the second user. Further, the intention of the second user is correlated with the current physiological state of the first user using user's biometrics, wherein the correlation includes variations in the physiological state of the first user captured by the physical activity sensor module (e.g., 102 of FIG. 2) and a brain activity sensor module (e.g., 103 of FIG. 2).

In step (204), the presence of an abnormality in the physiological state of the first user is determined by the processing module (e.g., 104 of FIG. 2), wherein the processing module (104) recognizes the physical activity of the first user by an activity detection unit (e.g., 105 of FIG. 2) and identifies the active attention state of the first user with respect to the transaction by a focused attention unit (e.g., 106 of FIG. 2.), where the Electroencephalograph's (EEG)/Magnetoencephalograph's (MEG) data collected from the brain activity sensor module (e.g., 103 of FIG. 2) is evaluated. An emotion detection unit (e.g., 107 of FIG. 2) identifies the physiological state of the first user to perform emotion classification and further, a disorder detection unit (108) identifies the presence of disorder in the first user using the Electroencephalograph's (EEG)/Magnetoencephalograph's (MEG) data collected from the brain activity sensor module (103).

The transaction is classified by a transaction classifier (e.g. 110 of FIG. 2) to determine if the user is safe to complete the transaction. In step (205), a recommendation module (e.g., 111 of FIG. 2) recommends the first user to focus on the transaction and prompts the first user for physical verification of the transaction using the method (200). According to an embodiment of the disclosure, the physical verification of the transaction may be One-time password, two-step verification, prompt authentication, fingerprint authentication etc. Further, in an embodiment, artificial intelligence and deep neural networks may be used for determination of the physiological state of the first user and intention of the second user.

FIG. 2 is a block diagram illustrating an example configuration of a system to verify transactions in the metaverse environment according to various embodiments. The system (100) may be implemented in an electronic device. The system (100) comprises an activator module (101) for monitoring initiation of a transaction from the first user. The activator module (101) checks whenever the transaction is initiated in the metaverse environment. Upon detection of the transaction initiation, the physical activity sensor module (102) captures the biometric data of the first user for activity recognition. The physical activity sensor module (102) comprises at least one sensor for recognizing the physical activity of the first user. Further, a brain activity sensor module (103) monitors the brain activity to identify the physiological state of the first user, wherein the brain activity sensor module (103) captures and processes the brain waves. The brain activity sensor module (103) comprises at least one sensor to capture the brain activity, wherein the brain activity sensor module (103) measures the Electroencephalograph's (EEG) signal from the electrical activity of the brain and the Magnetoencephalograph's (MEG) signal from the magnetic activity of the brain. Each of the modules include various circuitry and/or executable program instructions.

Further, the captured physical activity data and the brain activity data of the first user are processed by a processing module (104), e.g., including various processing circuitry, wherein the processing module (104) further comprises (i) an activity detection unit (105) for recognizing the presence of any physical activity from the first user, (ii) a focused attention unit (106) for identifying the active attention state of the first user with respect to the transaction, (iii) an emotion detection unit (107) for identifying the physiological state of the first user to perform emotion classification, and (iv) a disorder detection unit (108) for identifying the presence of disorder in the first user. Each of these units may include various circuitry and/or executable program instructions.

The physical activity data, active attention state and the disorder data processed by the processing module (104) is correlated with the intention of the second user interacting with the first user, wherein the intention of the second user is determined by an intention finder module (109). The intention finder module (109) determines the sentiments of the second user using speech signals. Further, based on the intention of the second user and the physiological data of the first user, a transaction classifier (110) classifies the initiated transaction to determine the authenticity of the transaction. Each of these modules may include various circuitry and/or executable program instructions.

Upon determination of the authenticity of the initiated transaction, a recommendation module (111) provides feedback to the first user and recommends the user to focus on the transaction. According to an embodiment of the disclosure, the feedback may be a neuro-feedback. Further, subsequent to recommendation, the recommendation module (111) prompts the first user to focus on the transaction by carrying out physical verification of the transaction. The recommendation module may include various circuitry and/or executable program instructions.

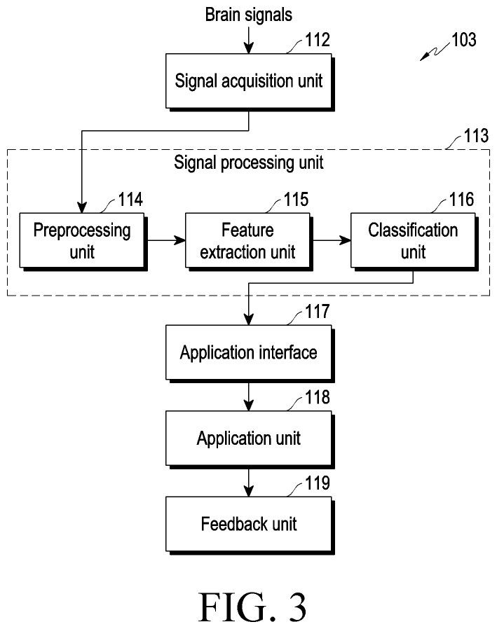

FIG. 3 is a block diagram illustrating an example configuration of the brain activity sensor module (103), according to various embodiments. A signal acquisition unit (112) acquires brain signals from the first user. The acquired brain signals, Electroencephalograph's e.g., (EEG) signals and Magnetoencephalograph's (MEG) signals are processed by a signal processing unit (e.g., including processing circuitry) (113), wherein the signal processing unit (113) further comprises a preprocessing unit (e.g., including various circuitry) (114) to preprocess the acquired Electroencephalograph's (EEG) signals and Magnetoencephalograph's (MEG) signals and a feature extraction unit (e.g., including various circuitry and/or executable program instructions) (115) for extracting the features from the brain signals. According to an embodiment of the disclosure, the Electroencephalograph (EEG) signals are captured by an Electroencephalograph (EEG) machine to track the electrical activity of the brain through the placement of electrodes on the scalp.

A classification unit (116) classifies the extracted features using artificial neural network to detect the required data, wherein the output from the classification unit (116) is provided to an application interface (117). The application interface (117) processes the classified data from the classification unit (116) with the help of an application unit (118), wherein the application unit (118) further comprises computational programs for processing the data, and a feedback unit (119) provides feedback to the first user based on the processed data.

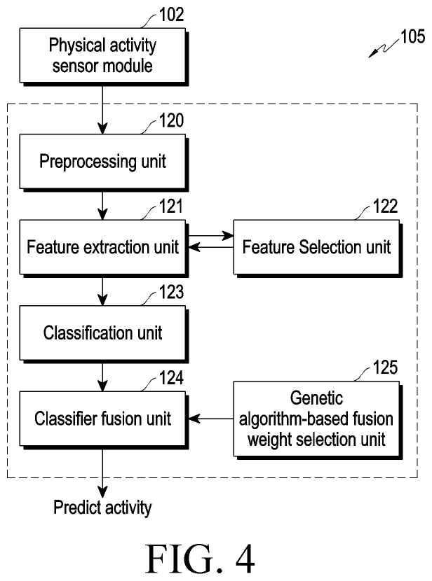

FIG. 4 is a block diagram illustrating an example of the activity recognition by the activity detection unit (105), according to various embodiments. The physical activity sensor module (102) comprises at least one sensor for detection of one or more type of physical activity, wherein multiple sensors including but not limited to accelerometer, gyroscope, infrared sensor, heart rate monitor and Photoplethysmography sensor are employed.

Further, the physical activity sensor module (102) measures the heart rate data using multiple sensors to boost classification of activities with diverse heart rates of the first user. The physical activity data captured by the physical activity sensor module (102) is preprocessed by a preprocessing unit (120) where the raw sensor data is extracted using 3D acceleration, heart rate, temperature, oxygen and 3D rotation captured by the sensors of the physical activity sensor module (102). Furthermore, a feature extraction unit (121) carries out feature extraction, wherein the feature extraction is facilitated by a feature selection unit (122) using forward checking. In an embodiment, the feature selection is based on the neural network and clamping technique in order to measure the impact of the clamped features within the network, wherein the feature selection unit (122) selects a group of features, which as a whole provides the best result.

The activity detection unit (105) further comprises a classification unit (123), wherein the classification unit (123) uses the selected group of features and classifies them using artificial neural networks including by not limited to Support Vector Machine (SVM), Multilayer Perceptron (MLP) neural network and Radial Basis Function (RBF) neural network. Further, in an embodiment, the artificial neural networks facilitate sensor-based activity recognition, wherein the Support Vector Machine (SVM) constructs decision boundaries by solving the optimization objective and performs multiclass classification using K-binary classifiers and one-vs-all classifiers.

Further, a classifier fusion unit (124) incorporates weights into the classification model of the classification unit (123), wherein the classifier fusion unit (124) uses a genetic algorithm-based fusion weight selection (GAFW) approach by a Genetic algorithm-based fusion weight selection unit (125) to find the fusion weights. The genetic algorithm-based fusion weight selection (GAFW) approach uses a genetic algorithm to find fusion weights for classifiers in order to optimize the classified data, wherein the genetic algorithm creates a population of points where the population is modified over time. The classifier fusion unit (124) further predicts the physical activity of the user with the help of the data from the Genetic algorithm-based fusion weight selection unit (125). Each of the units described above may include various circuitry and/or executable program instructions.

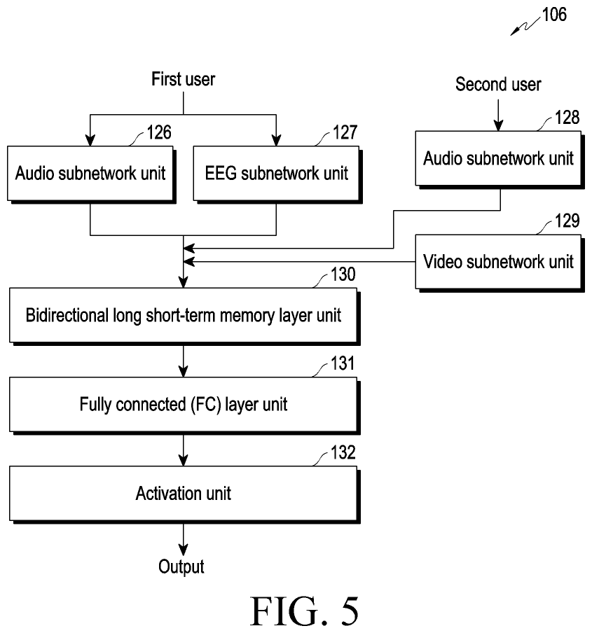

FIG. 5 is a block diagram illustrating an example configuration of the focused attention unit, according to various embodiments. The focused attention unit (106) comprises an audio subnetwork unit (126) to process the speech signals captured from the first user, wherein according to an embodiment, the audio subnetwork unit (126) uses at least one layer of Convolutional Neural Network (CNN) to process the speech spectrogram of the first user. The audio subnetwork unit (126) processes the data from the Convolutional Neural Network (CNN) layers using pooling layers and fully connected layers for classifying the audio data of the first user.

The focused attention unit (106) further comprises an EEG subnetwork unit (127) to process the Electroencephalograph's (EEG) signals captured from the first user through Electroencephalogram (EEG), wherein Electroencephalogram (EEG) is a test to measure the electrical activity of the brain. According to an embodiment of the disclosure, the EEG subnetwork unit (127) may comprise at least one Convolutional Neural Network (CNN) layer to process the Electroencephalograph's (EEG) signal of the first user, wherein the data from the Convolutional Neural Network (CNN) layers is passed through the pooling layer and subsequently, the output is passed through a non-linear activation function known as a rectified linear unit (ReLU).

Further, an audio subnetwork unit (128) processes the speech signals captured from the second user, wherein the audio subnetwork unit (128) uses at least one layer of Convolutional Neural Network (CNN) to process the speech spectrogram according to an embodiment. The data from the Convolutional Neural Network (CNN) layers are passed through at least one pooling layer and a fully connected layer to classify the audio data of the second user. Further, a video subnetwork unit (129) may use Stacked Attention Networks (SAN) to process the video data in the metaverse environment according to an embodiment of the disclosure. The Stacked Attention Network (SAN) further comprises an image model, a question model, and a stacked attention model, wherein the stacked attention model locates the image regions in the captured video that are relevant to the question for answer prediction. With the help of the image mode and the question model, the Stacked Attention Network (SAN) of the video subnetwork unit (129) predicts the answer through multi-step reasoning and gradually filters the noises to focus on the relevant regions for determination of focus state of the first user.

The focused attention unit (106) further comprises a Bidirectional long short-term memory layer (LSTM) unit (130) to process the feature maps obtained from the audio subnetwork unit (126), EEG subnetwork unit (127), audio subnetwork unit (128) and the video subnetwork unit (129) according to an embodiment of the disclosure, wherein the Bidirectional long short-term memory layer (LSTM) unit (130) processes the concatenated feature maps and the output data is passed through at least one fully connected layer in the Fully Connected (FC) layer unit (131). The Fully Connected (FC) layer unit (131) further comprises at least one Fully Connected (FC) layer for data processing and ReLU activation is used in the Fully Connected (FC) layer. Further, an activation unit (132) may use SoftMax activation on the output data to classify the attention to the speaker according to an embodiment of the disclosure. Each of the units described above may include various circuitry and/or executable program instructions.

According to an example embodiment of the present disclosure, the focused attention unit (106) uses a joint CNN-LSTM model to process the speech signals and EEG signals of the first user, speech signals of the second user, and input video frame, wherein the focused attention unit (106) determines the attention of the user to confirm if the attention is focused on the transaction. The focused attention unit (106) creates a relationship between the speech signals of the first user and the second user, video frame and the Electroencephalograph's (EEG) signal of the first user and quantitatively evaluates the processed data. Further, the focused attention unit (106) integrates the feedback into an electronic device of the first user, wherein the electronic device infers the attention state of the first user.

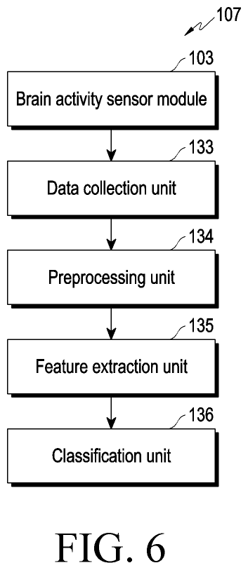

FIG. 6 is a block diagram illustrating an example configuration of the emotion detection unit, according to various embodiments. The emotion detection unit (107) comprises a data collection unit (133) to collect the Electroencephalograph's (EEG) signals from the first user captured by the brain activity sensor module (103). The Electroencephalograph's (EEG) signal data from the data collection unit (133) is processed by a preprocessing unit (134), and a feature extraction unit (135) extracts the features from the preprocessed data into frequency bands. The frequency bands containing the extracted features may include delta waves, theta waves, alpha waves, beta waves and gamma waves. The extracted features are classified into various emotions by a classification unit (136). Each of the units described above may include various circuitry and/or executable program instructions.

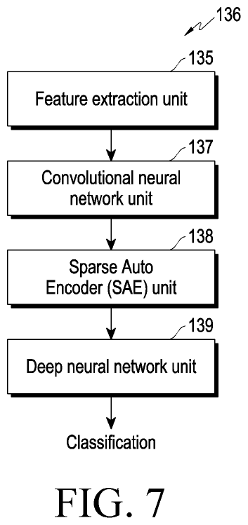

FIG. 7 is a block diagram illustrating an example configuration of the classification unit of the emotion detection unit, according to various embodiments. The the classification unit (136) uses at least one neural network including, but not limited to Convolutional Neural Network (CNN), Sparse Autoencoder (SAE), and Deep Neural Network (DNN), wherein the feature extraction unit (135) extracts the features from the preprocessed data. A Convolutional Neural Network (CNN) unit (137) further comprises at least one Convolutional layer and a max-pooling layer, wherein the data from the pre-processing unit (134) passes through at least one Convolutional layer of the Convolutional Neural Network (CNN) unit (137), where dropout connects to each convolutional layer. The processed data is pooled and flattened and processed through a Sparse Autoencoder (SAE) of the Sparse Autoencoder (SAE) unit (138), wherein the Sparse Autoencoder (SAE) unit (138) further comprises an encoding layer, a hidden layer, and a decoding layer. The Sparse Autoencoder unit (SAE) (138) preserves the essence of the input data and removes the potential noise in the data in an unsupervised manner by dividing the data processing method into encoding and decoding phases. Further, the output data from the Sparse Autoencoder unit (SAE) (138) is processed by a Deep Neural Network unit (139).

A classification unit (136) classifies the extracted features into types of emotion, wherein the emotion of the first user is determined by the classification unit (136). In an embodiment, the classification unit (136) uses Deep Neural Network (DNN) from the Deep Neural Network (DNN) unit (139) for data classification, wherein the Deep Neural Network (DNN) further comprises at least one fully connected layer and the fully connected layers uses ReLU activation function, facilitating the classification of data into various emotions. Each of the units described above may include various circuitry and/or executable program instructions.

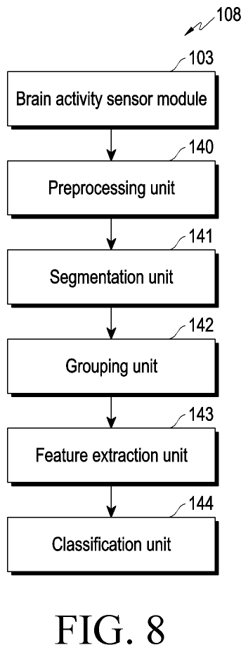

FIG. 8 is a block diagram illustrating an example configuration of the disorder detection unit, according to various embodiments. The disorder detection unit (108) further comprises a preprocessing unit (140) to process the Electroencephalograph's (EEG) signals of the first user captured by the brain activity sensor module (103). According to an embodiment of the disclosure, the Electroencephalograph's (EEG) signals are filtered and then segmented into sleep stages according to annotations of sleep stages in the database by a segmentation unit (141), wherein an Electroencephalograph (EEG) epoch e.g., a 30 second duration of the Electroencephalograph's (EEG) signal is labeled with a sleep stage. Further, a grouping unit (142) groups the Electroencephalograph (EEG) epochs into various sleep stages, wherein wavelet decomposition of Electroencephalograph's (EEG) signals is performed using triplet half-band filter pair and various sub-bands corresponding to each Electroencephalograph (EEG) epoch are obtained.

Further, a feature extraction unit (143) is used to extract the Hjorth parameters including activity, mobility, and complexity from each sub-band, and a classification unit (144) processes the extracted Hjorth parameters using various supervised machine learning classifiers for automated detection of the type of sleep disorder in the first user. According to an embodiment of the disclosure, Electroencephalograph's (EEG) signals facilitate detection and classification of several brain related disorders, wherein the disorders can be any disorder including sleep disorder, mental illness etc. Each of the units described above may include various circuitry and/or executable program instructions.

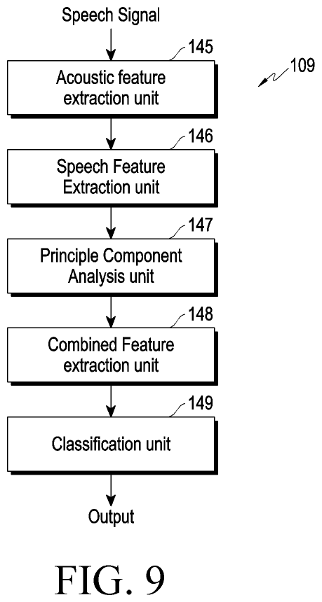

FIG. 9 is a block diagram illustrating an example configuration of the intention finder module, according to various embodiments. The intention finder module (109) determines the intention of the second user where the sentiments are identified through interpolating and extrapolating speech by a speech model. The speech signals from the second user are captured and the acoustic features of the second user's voice are extracted from the raw audio input by an acoustic feature extraction unit (145), wherein extracted raw audio signals from the second user are processed by a speech feature extraction unit (146). Further, the speech feature extraction unit (146) comprises at least one speech feature extraction model including Lincar Predictive Cepstral Coefficients (LPCC), and Mel frequency Cepstrum Coefficient. (MFCC), and Gamma tone Frequency Cepstral Coefficients (GFCC). The speech feature extraction models process the raw audio input and a Principal Component Analysis (PCA) (147) performs speech data reduction to identify the combined features.

Further, a combined feature extraction unit (148) processes the identified combined features, wherein a speech intention algorithm uses real-time intention finders to identify the sentiments of packets and for each lost frame, a deep model estimates the features of the lost frame and overlap them to audio content. The combined feature extraction unit (148) comprises a Convolutional Neural Network (CNN) model, wherein the CNN model further comprises of a pair of convolutional and pooling layers, with at least one convolutional filter with a ReLU activation layer according to an embodiment of the disclosure. The data that passes through the convolutional layer is further passed through a MaxPool layer, wherein the output data from the MaxPool layer passes through a flattening layer and a series of fully connected layers with a rectifier activation function and SoftMax activation function. The output data from the combined feature extraction unit (148) is further classified by a classification unit (149), wherein the classification unit (149) classifies the processed combined features to classify them into different emotions, wherein the classification unit (149) determines the sentiments of the second user by interpolating and extrapolating the speech data. The various units and modules described above may include various circuitry and/or executable program instructions.

According to the present disclosure, the intention of the second user determined by the intention finder module (109) and the data of the first user processed by the processing module (104) is used by a transaction classifier (110) to classify the transaction, wherein at least one physiological parameter of the first user and the intent of the second user determines the authenticity of the transaction. The transaction classifier (110) uses 5D parameter classification wherein the emotions, productivity, sleep, physical activity, and intention of the second user are used by the binary classifier to facilitate the classification of the transaction.

According to an embodiment of the disclosure, the binary classification is carried out by a K-Nearest neighbor (K-NN) classifier to determine the authenticity of the transaction, wherein the K-NN classifier is a non-parametric supervised learning classifier for selecting the number K of the neighbors and calculating the Euclidean distance of K number of neighbors. By considering the K nearest neighbors as per the calculated Euclidean distance, the number of data points in each category is calculated among the K neighbors and new data points are assigned to that category having the highest number of the neighbor. According to an embodiment of the disclosure, the transaction classified by the transaction classifier (110) may be a monetary transaction, a meeting or an exchange of objects.

Further, the recommendation module (111) provides feedback to the first user based on the output of the transaction classifier (110) and recommends the first user to focus on the transaction. The recommendation module (111) provides alerts to the first user in case of abnormalities in the physiological state and recommends accompanying of at least one user with the first user for attentive action. According to an embodiment of the disclosure, the abnormalities may include social abnormalities, good/bad touch etc. Further, the recommendation module (111) provides recommendation to the first user to perform physical verification to complete the transaction.

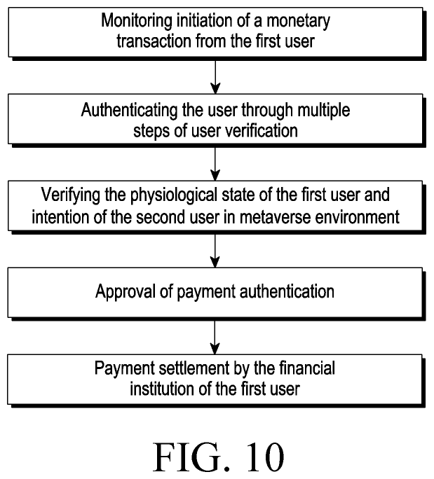

FIG. 10 is a flowchart illustrating an example first use case pertaining to a monetary transaction performed by the first user through an electronic device in a metaverse environment according to various embodiments. Consider a transaction initiated by the first user, wherein the activator module (101) monitors the initiated monetary transaction and the first user is authenticated by at least one step of verification including, but not limited to fingerprint verification, password, and One-time-password. Upon verification of the first user, the system (100) and the method (200) determine the physiological state of the first user through the processing module (104) and the intention of the second user interacting with the first user through the intention finder module (109) to validate the second user in the metaverse environment. Further, based on the physiological state of the first user and the intention of the second user, the transaction classifier (110) classifies the transaction as safe to perform by determining its authenticity. Further, upon classifying the transaction as safe by the transaction classifier (110) and if the attention of the first user is on the transaction, the first user completes the transaction and the transaction is approved. Hence, based on the above-mentioned estimations, the payment is settled by the financial institution of the first user upon receiving approval of the transaction completion process. Therefore, it is apparent that the system (100) and method (200) overcome the challenges of the prior art by providing an accurate payment system wherein the first user and second user are validated for completing a monetary transaction.

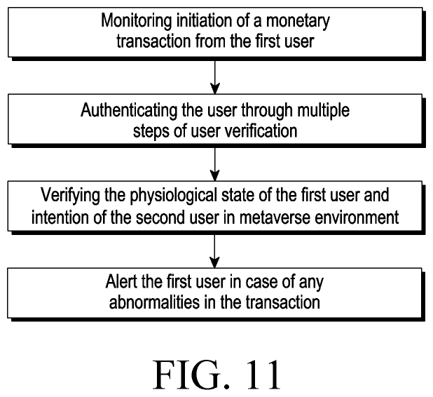

FIG. 11 is a flowchart illustrating an example second use case pertaining to a wrong transaction where a monetary transaction is performed by the first user to a wrong bank account or wallet according to various embodiments. Consider a monetary transaction initiated by the first user, wherein the first user sends a certain amount to a wrong bank account or wallet through an online transaction in a stressful situation. The activator module (101) monitors the initiated monetary transaction and the first user is authenticated by at least one step of verification including, but not limited to fingerprint verification, password and One-time-password. Upon verification of the first user, the system (100) and the method (200) determine the physiological state of the first user through the processing module (104) and the intention of the second user interacting with the first user through the intention finder module (109) to validate the second user in the metaverse environment. Further, the transaction classifier (110) classifies the transaction and the recommendation module (111) alerts the first user in case of abnormalities and informs the user that there are abnormalities in the initiated monetary transaction. Therefore, it is apparent that the system (100) and method (200) overcomes the challenges of the prior art by providing an accurate payment system where the first user is alerted when a wrong monetary transaction is initiated.

Embodiments of the disclosure provide a system (100) and method (200) for verifying a transaction in the metaverse environment to prevent and/or reduce the physiological tricks on the first user, therefore, overcoming the lack of first user and second user validation provided by the existing transaction verification systems. The processing module (104) provided in the system (100) determines the physiological state of the first user and the intention finder module (109) determines the intention of the second user in order to verify the transaction.

Further, the system (100) and method (200) provide a secure environment for the first user to perform the transactions in the virtual environment by providing an additional layer of security by verifying the transaction. The focused attention unit (106) provided in the system (100) determines if the attention of the first user is on the initiated transaction. The recommendation module (111) alerts the first user in case of abnormalities in the transaction and recommends the first user to provide attentive action, wherein the recommendation module (111) further blocks the transaction if required.

According to various example embodiments of the present disclosure, the intention of the second user interacting with the first user is determined using an intention finder module, wherein the speaker sentiment of the second user is detected using speech models. The system classifies the transaction using a transaction classifier based on the intention of the second user and at least one parameter of the first user derived from the processing module, wherein the transaction classifier determines the authenticity of the transaction. Further, a recommendation module provides feedback to the first user and recommends the first user to focus on the transaction including physical verification for the transaction. The feedback provided by the recommendation module is integrated into any electronic device used by the first user, wherein the electronic device deduces the attention of the first user.

Thus, the present disclosure provides a system and method for verifying transactions between a first user and a second user in a metaverse environment, where the physical activity of the first user is determined by the physical activity sensor module using at least one sensor for detection of one or more type of physical activity and the brain activity of the first user is determined by the brain activity sensor module using at least one sensor for detection of one or more type of brain waves to measure the Electroencephalograph's (EEG) signal and Magnetoencephalograph's (MEG) signal.

The physical activity data captured by the physical activity sensor module is processed by the processing module, wherein by quantitatively evaluating Electroencephalograph's (EEG) signal and Magnetoencephalograph's (MEG) signal by a focused attention unit, the active attention state of the first user with respect to the transaction is identified. Further, the Electroencephalograph's (EEG)/Magnetoencephalograph's (MEG) data is processed to extract the features and perform emotion classification by an emotion detection unit and to identify the presence of any disorder by a disorder detection unit. The emotional state of the first user is determined by the emotion detection unit, wherein the Electroencephalograph's (EEG) data is preprocessed and the features are extracted in frequency bands, wherein the extracted features are classified into emotions. The data processed by the processing module and the intention of the second user are used for the classification of the transaction by a transaction classifier.

At least one of the plurality of modules may be implemented through an AI model. Functions of the plurality of modules and the function associated with AI may be performed through the non-volatile memory, the volatile memory, and the processor of the electronic device. The processor may include one or a plurality of processors. One or a plurality of processors may be a general-purpose processor, such as a central processing unit (CPU), an application processor (AP), or the like, a graphics-only processing unit such as a graphics processing unit (GPU), a visual processing unit (VPU), and/or an AI-dedicated processor such as a neural processing unit (NPU). The processor may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The one or a plurality of processors control the processing of the input data in accordance with a predefined operating rule or artificial intelligence (AI) model stored in the non-volatile memory and the volatile memory. The predefined operating rule or artificial intelligence model is provided through training or learning. Here, being provided through learning may refer, for example to, by applying a learning algorithm to a plurality of learning data, a predefined operating rule or Al model of a desired characteristic being made. The learning may be performed in a device itself in which Al according to an embodiment is performed, and/or may be implemented through a separate server/system.

The AI model may include a plurality of neural network layers. Each layer has a plurality of weight values, and performs a layer operation through calculation of a previous layer and an operation of a plurality of weights. Examples of neural networks include, but are not limited to, convolutional neural network (CNN), deep neural network (DNN), recurrent neural network (RNN), restricted Boltzmann Machine (RBM), deep belief network (DBN), bidirectional recurrent deep neural network (BRDNN), generative adversarial networks (GAN), and deep Q-networks. The learning algorithm is a method for training a predetermined target device (for example, a robot) using a plurality of learning data to cause, allow, or control the target device to make a determination or prediction. Examples of learning algorithms include, but are not limited to, supervised learning, unsupervised learning, semi-supervised learning, or reinforcement learning.

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

What is claimed is:

1. A method for verifying transactions in a metaverse environment, the method comprising:
detecting a first user involved in an activity using user biometrics, wherein the first user is in focused attention state or addiction state;
identifying a second user interacting with the first user during a transaction;
identifying an intention of the second user interacting with the first user;
determining a presence of an abnormality in a physiological state of the first user; and
recommending to the first user to focus on the transaction.

2. The method of claim 1, wherein the intention of the second user is correlated with the physiological state of the first user, where the correlation includes variations in the physiological state of the first user comprising a physical activity and a brain activity of the first user.

3. The method of claim 2, further comprising detecting the brain activity of the first user by measuring one or more types of brain waves including at least one of Electroencephalograph's (EEG) signal from electrical activity of a brain of the first user and Magnetoencephalograph's (MEG) signal from magnetic activity of the brain of the first user.

4. The method of claim 1, wherein determining the presence of the abnormality in the physiological state of the first user comprises:
recognizing a physical activity of the first user, deriving a plurality parameters of the first user from biometric data of the first user, and processing the plurality of parameters using a neural network;
identifying an active attention state of the first user with respect to the transaction, by quantitatively evaluating Electroencephalograph's (EEG) data and Magnetoencephalograph's (MEG) data collected from a brain of the first user;
identifying the physiological state of the first user, wherein the EEG data and the MEG data are processed to extract features and perform emotion classification; and
identifying a presence of disorder in the first user based on the EEG data and the MEG.

5. The method of claim 4, wherein recognizing the physical activity of the first user comprises:
pre-processing raw sensor data extracted from at least one sensor configured to detect the biometric data of the first user; and
selecting features based on the pre-processed raw sensor data; and
classifying the selected features to achieve sensor-based activity recognition.

6. The method of claim 4, wherein identifying the active attention state of the first user with respect to the transaction comprises:
obtaining speech signals of the first user, speech signals of the second user, a video frame of the metaverse environment and the EEG data of the first user; and
creating relationship between the speech signals of the first user, the speech signals of the second user, the video frame of the metaverse environment and the EEG data of the first user.

7. The method of claim 1, further comprising:
generating feedback including neuro-feedback derived from brain signals of the first user,
wherein the generated feedback is integrated into an electronic device used by the first user to deduce the attention of the first user based on the feedback.

8. The method of claim 4, wherein performing the emotion classification comprises:
preprocessing raw Electroencephalograph's (EEG) data captured from the brain of the first user; and
extracting features from the preprocessed raw EEG data; and
classifying the extracted features to classify an emotional state of the first user.

9. The method of claim 4, wherein identifying a presence of disorder in the first user comprises:
capturing EEG signals of the first user;
filtering and classifying the EEG signals into a plurality of sleep stages according to annotations of the sleep stages in a database, where an EEG epoch is labeled with a sleep stage;
preprocessing the EEG signals;
performing wavelet decomposition of the preprocessed EEG signals to obtain a plurality of sub bands corresponding to each EEG epoch;
extracting Hjorth parameters from each sub band; and
processing the extracted Hjorth parameters using a plurality of classifiers for detecting of a type of a sleep disorder in the first user.

10. The method of claim 8, wherein identifying a presence of disorder in the first user further comprises:
classifying the EEG signals into specified sleep models.

11. The method of claim 1, wherein identifying the intention of the second user interacting with the first user comprises:
identifying sentiments of the second user by interpolating and extrapolating speech signals of the second user using a speech model.

12. The method of claim 1, further comprising:
classifying the transaction to determine an authenticity of the transaction based on at least one physiological parameter of the first user and the intention of the second user.

13. The method of claim 1, further comprising:
providing alerts to the first user based on abnormalities in the physiological state; and
recommending to accompany at least one user with the first user for attentive action.

14. The method of claim 1, further comprising:
providing recommendation to the first user to perform physical verification to complete the transaction.

15. An electronic device configured to verify transactions in the metaverse environment, the electronic device comprising:
a memory; and
at least one processor, comprising processing circuitry, coupled to the memory, wherein the at least one processor, individually and/or collectively, is configured to:
detect a first user involved in an activity using user biometrics, wherein the first user is in focused attention state or addiction state,
identify a second user interacting with the first user during a transaction,
identify an intention of the second user interacting with the first user,
determine a presence of an abnormality in a physiological state of the first user, and
recommend to the first user to focus on the transaction.

16. The electronic device of claim 15, wherein the intention of the second user is correlated with the physiological state of the first user, where the correlation includes variations in the physiological state of the first user comprising a physical activity and a brain activity of the first user.

17. The electronic device of claim 16, wherein the at least one processor, individually and/or collectively, is further configured to detect the brain activity of the first user by measuring one or more types of brain waves including at least one of Electroencephalograph's (EEG) signal from electrical activity of a brain of the first user and Magnetoencephalograph's (MEG) signal from magnetic activity of the brain of the first user.

18. The electronic device of claim 15, wherein to determine the presence of the abnormality in the physiological state of the first user, the at least one processor, individually and/or collectively, is configured to:
recognize a physical activity of the first user, deriving a plurality parameters of the first user from biometric data of the first user, and processing the plurality of parameters using a neural network;
identify an active attention state of the first user with respect to the transaction, by quantitatively evaluating Electroencephalograph's (EEG) data and Magnetoencephalograph's (MEG) data collected from a brain of the first user;
identify the physiological state of the first user, wherein the EEG data and the MEG data are processed to extract features and perform emotion classification; and
identify a presence of disorder in the first user based on the EEG data and the MEG.

19. The electronic device of claim 18, wherein to recognize the physical activity of the first user, the at least one processor, individually and/or collectively, is configured to:
pre-process raw sensor data extracted from at least one sensor configured to detect the biometric data of the first user; and
select features based on the pre-processed raw sensor data; and
classify the selected features to achieve sensor-based activity recognition.

20. The electronic device of claim 18, wherein to identify the active attention state of the first user with respect to the transaction, the at least one processor, individually and/or collectively, is configured to:
obtain speech signals of the first user, speech signals of the second user, a video frame of the metaverse environment and the EEG data of the first user; and
create relationship between the speech signals of the first user, the speech signals of the second user, the video frame of the metaverse environment and the EEG data of the first user.
